(19) United States
(12) Patent Application Publication
(10) Pub. No.: US 2008/0060658 A1
(43) Pub. Date: Mar. 13, 2008

(54) Blocking Device for Fallopian Tubes and Introduction Device Therefor

(75) Inventor: Benedict Marie Doorschodt, Amsterdam (NL)

Correspondence Address: THE WEBB LAW FIRM, P.C., 700 KOPPERS BUILDING, 436 SEVENTH AVENUE, PITTSBURGH, PA, 15219, US

(73) Assignee: DOORZAND GUARDIAN ANGEL B.V., AMSTERDAM (NL)

(21) Appl. No.: 11/579,436

(22) PCT Filed: May 3, 2005
(86) PCT No.: PCT/NL05/00338

§ 371 Date: Aug. 7, 2007

(30) Foreign Application Priority Data
May 3, 2004 (NL) 1026102

(51) Int. Cl. A61F 6/06

(52) U.S. Cl. 128/831; 606/191

(57) ABSTRACT

The invention relates to a blocking device for a Fallopian tube and introduction device thereof. The blocking device consists of a central section with adjoining elongated retaining means on both sides thereof which are displaceable between an introduction position and a retaining position. In the introduction position, the retaining means are in line with the central section. The introduction device consists of a construction comprising a handle, central bush and sliding bush, as a result of which it is possible, on the one hand, to displace the blocking device over a defined distance accommodated inside an introduction sleeve when it comes into contact with the constriction of the Fallopian tube, and, on the other hand, to allow the blocking device to unfold without further displacement after it has been fitted in the correct position.

The present invention relates to a blocking device comprising a central section and two elongated retaining means attached on both sides thereof and resiliently displaceable between an introduction position and a position of use, said retaining means being in line with the central section in the introduction position. A blocking device of this type is known from U.S. Pat. No. 5,669,933. The purpose of the blocking device described in the latter is to introduce the blocking device in a vascular passageway once. The retaining means are arranged on both ends of the central section. The retaining means at the one end can be folded up in line with the central section. The retaining means at the other end can be folded up overlapping the central section. The latter retaining means are curved away from the central section.

U.S. Pat. No. 4,595,000 A describes a blocking device for Fallopian tubes comprising a central section. In order to keep a blocking device in position in the Fallopian tube, resilient barbs are provided near the free end thereof, which barbs act as retaining means. These enable displacement in one direction, but block displacement in the opposite direction by folding out.

As the central section has to be designed with as small a diameter as possible, such retaining means will also have to be designed to be relatively small. By the nature of their design, these retaining means form sharp protruding parts, which may either cause damage to the Fallopian tube or cause great discomfort.

FR 2641692 discloses a great variety of blocking devices for all sorts of applications. Devices which are introduced with tools consist of a simple wad.

It is an object of the present invention to provide a blocking device with retaining means which are designed such that, on the one hand, displacement of the blocking device can be prevented in an effective and secure manner and, on the other hand, the wearing comfort is optimized. In addition, a blocking device of this type should be easy to fit, that is to say the retaining means should hardly increase the cross-sectional dimensions of the blocking device, if at all.

This object is achieved in an above-described blocking device in that said blocking device is embodied for use in a Fallopian tube and that the retaining means comprise arcuate parts which are curved towards the central section in the position of use.

It has been found that upon introduction of the blocking device, the length thereof is less relevant than the cross-sectional dimensions. Because, according to subject invention, the retaining means are now placed in line with the central section in the introduction position, these can be embodied to be of relatively large dimension. After all, as long as the dimensions of the retaining means (in cross section) do not exceed those of the central section (in cross section), there will be no problems during introduction. As a result, there is more freedom to design the retaining means in such a manner that they, on the one hand, ensure that they always maintain the blocking device in position and, on the other hand, they can be designed to be as “comfortable” as possible. This also means that when it is desired to remove the blocking device, this can be done without much damage to the Fallopian tube.

According to an advantageous embodiment of the invention the retaining means comprise arcuate parts which are curved towards the central section in the operating state. That is to say, the retaining means extend from the central section in the manner of an adjoining arc, the free end of the arc (all this in the operating state) being directed back at the central section (and not to the continuation thereof).

It is possible to attach one or more retaining means on one or both ends of the central section. Preferably, however, each end is provided with two opposite retaining means.

Both the retaining means and the central section may consist of wire, such as a silver wire. In one preferred embodiment, the retaining means are provided with a flexible coating in order to increase the wearing comfort. This coating preferably comprises silicone-like material. It is also possible to coat the central section with material of this type.

According to a preferred embodiment of the invention, at least the central section consists of a wire, around which a relatively soft material is fitted, as has been described above. Through a combination of the particular shape of the retaining means and the soft material used, which can preferably also be used in the retaining means, it can be guaranteed, on the one hand, that the blocking device maintains its position in the Fallopian tube after its introduction and, on the other hand, it can be guaranteed that it can be removed therefrom without damaging the Fallopian tube. By using a wire or another high-strength part around which the soft material is fitted in the central section, it is possible to prevent remains from being left behind in the Fallopian tube. This can furthermore be insured by also constructing the retaining means in a similar manner, that is to say provided with a central section of a relatively high strength, such as a wire, around which soft material is fitted. Such a central part in the retaining means can also provide the resilience of the retaining means.

In addition, the blocking device is preferably provided with a loop for the subsequent removal thereof. This loop is arranged near one of the ends of the blocking device, preferably as a continuation of the central section.

The present invention also relates to an introduction device for fitting the blocking device described above. This introduction device comprises an introduction sleeve with an introduction wire which can be displaced therein, the introduction wire being displaceable within this introduction sleeve from a position in which a space for the blocking device is delimited in front of this wire at the introduction end in this introduction sleeve to a position in which the free end of this wire is substantially level with the free end of this introduction sleeve.

According to the present invention, a blocking device is fitted, arranged in the free end of an introduction sleeve. An introduction wire is present in this introduction sleeve, which introduction wire adjoins the blocking device from behind. According to the present invention, the introduction sleeve, introduction wire and blocking device are brought into the desired position within the Fallopian tube and the blocking device will be released from the introduction sleeve without being displaced in the direction of introduction by the introduction sleeve being withdrawn relative to the introduction wire and the blocking means will be able to unfold immediately.

In order to enable such a movement of the introduction sleeve and the introduction wire relative to one another, the introduction device may be provided with particular means, through which it is possible to withdraw the introduction sleeve over a defined distance, which corresponds to the length of the blocking device, relative to the introduction wire.

When the blocking device is being introduced, it is important that it is fitted in the Fallopian tube in the correct position. It has been found that there is a constriction in the Fallopian tube. This constriction can easily be detected with the introduction device described above as increased resistance will be experienced during the introduction procedure. According to the present invention, means are present in order to advance, starting from this position of increased resistance, the introduction sleeve, blocking device and introduction wire over a defined distance while maintaining their relative position with respect to one another. This defined distance is preferably between 1.5 and 2.5 cm. Thus, it is possible to determine exactly the positioning of the blocking device. All this can be achieved by using the constructions described below with reference to exemplary embodiments provided with handles, central bushes and sliding bushes.

The invention described above will be described in more detail below. Therein:

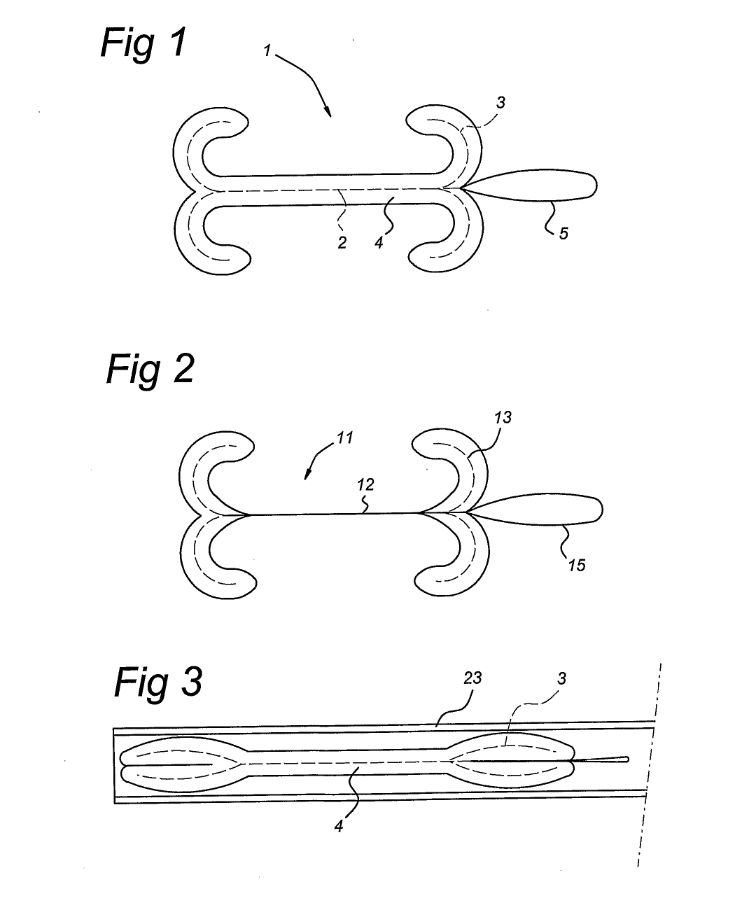

FIG. 1 shows a perspective view of the blocking unit according to the present invention in a first embodiment and in folded-out position;

FIG. 2 shows a second embodiment of the blocking unit according to the invention;

FIG. 3 shows the blocking unit according to the invention in the folded-in position;

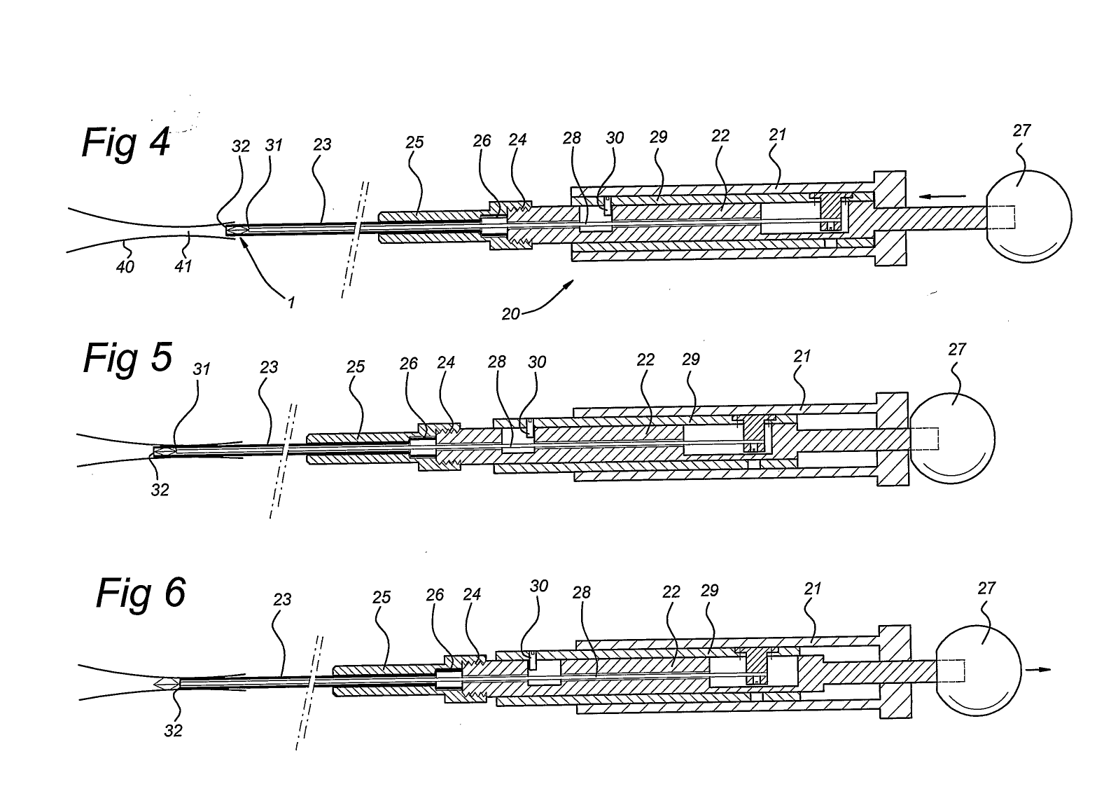

FIG. 4 shows the introduction device according to the invention in a first position;

FIG. 5 shows the introduction device according to the invention in a second position; and

FIG. 6 shows the introduction device according to the invention in a third position.

The blocking device according to the invention is denoted overall in FIG. 1 by reference numeral 1. It consists of a central wire 2, which branches into arc parts 3 on both sides. That is to say that the central section is situated between the arc parts. A coating made of silicone material 4 is present. As a result of the curved shape of the arcuate parts 3, it is possible to determine the optimum positioning in the Fallopian tube. A loop, preferably likewise made from a metal wire which does not react with the environment of the human body, is denoted by reference numeral 5. By means thereof, it is possible to remove the blocking device 1 after fitting.

In FIG. 2, an embodiment similar to the one of FIG. 1 is denoted overall by reference numeral 11. In this case, there is also a central silver wire 12 which extends towards the arcuate parts 13. In this embodiment, only the arcuate parts are provided with a coating, such as silicone material. This embodiment also has a pulling loop 15.

As FIG. 3 shows, this blocking device is stored inside a sleeve 23, further details of which are shown in FIGS. 5 et seq., the arc-shaped parts 3 and 13, respectively, being folded out, that is to say, in such a manner that they assume an outwardly folded position. As a result of the resilience, each of the arcuate parts 3 will spring back from the extended position to the position shown in FIGS. 1 and 2, respectively, when the blocking device is removed from the sleeve 23. The wire 12 may also comprise a spring.

FIGS. 4-6 show an example of a device for introducing the blocking device. This device is denoted overall by reference numeral 20 and consists of a handle 21, inside which a central bush 22 is arranged so as to be displaceable. This is provided with a screw thread 24 near the end, onto which an auxiliary bush 25 can be screwed. Sleeve 23 is an elongated flexible duct and provided with thickening 26 at the end. By screwing the end of the auxiliary bush 25 onto the screw thread 24, the thickening 26 is locked. That is to say, when the central bush 22 is moved in a reciprocating manner, the sleeve moves along to exactly the same degree. Pins are used to ensure that the auxiliary bush 22 cannot rotate with respect to a handle. The above-described reciprocating movement can be achieved by means of a button 27.

A central wire made of relatively stiff material is denoted by reference numeral 28. This can slide to and fro within sleeve 23, the end remote from the free end of the sleeve being attached to a sliding bush 29. This sliding bush 29 can execute a limited stroke relative to the handle 21 and relative to the central bush 22. Central bush 22 is provided with a stop 30 for the sliding bush 29.

In the position shown in FIG. 4, the button 27, together with the central bush 22, is pulled outwards relative to the handle 21. Sliding bush 29 is pushed inwards relative to the handle 21. As a result of this arrangement, the end 31 of the wire 28 is at some distance from the free end 32 of the sleeve 23. The free space is exactly sufficient for receiving, for example, a blocking device 1. On the left-hand side in FIG. 4, a Fallopian tube is shown which has a constricted end 41 at its outlet. In the position shown in FIG. 4, the sleeve 23 with wire 28 and blocking device 1 is introduced until the stop at the constriction 41 is felt.

Starting from this position, the button 27 is pushed in while the handle 21 is kept stationary. As a result, the bush 22, and therefore the sleeve 23, move to the left, past the constriction 41. As, during the pushing action described above, the sliding bush 29 comes to bear against the stop 30 of the central bush 22, the sliding bush 29 and therefore wire 28 are entrained. That is to say, the positioning of the wire 28 and the sleeve 23 relative to one another does not change, but as a result of button 27 being pushed in, the wire 28 and the sleeve 23 are displaced to the left over a defined distance, which corresponds to the distance of the button 27 and handle 21 shown in FIG. 4. This distance corresponds exactly to the desired displacement of the blocking device 1 relative to the constriction 41 of the Fallopian tube.

Subsequently, as is shown in FIG. 6, the button 27 is pulled back. Stop 30 is no longer active, so that this pulling back has no effect on the sliding bush 29, i.e. the wire is not affected during the pulling back. The sleeve 23, however, is affected, since it is coupled to the central bush 22. That is to say, as a result of the pulling-back motion of the button 27, the sleeve 23 moves back relative to wire 28 and the free end comes to lie level with the free end 32. As a result, the blocking device is uncovered and the arcuate parts 3 and 13, respectively, hinge back and fixing takes place.

All this is embodied in such a manner that the loop 5 described above is located outside the Fallopian tube, as a result of which subsequent removal of the device 1 will be easy.

Although the invention has been described above with reference to a preferred embodiment, it will be understood that many alterations can be made thereto. These will be immediately apparent to those skilled in the art upon reading the above description.

1-15. (canceled)

16. A blocking device, comprising a central section and elongated retaining means attached on both sides thereof and resiliently displaceable between an introduction position and a position of use, said retaining means being in line with the central section in the introduction position, said blocking device being embodied for use in a Fallopian tube and said retaining means comprising arcuate parts having free extremities and which are curved towards the central section in the position of use.

17. The blocking device according to claim 16, wherein said retaining means comprise two retaining means arranged on opposite sides of the central section.

18. The blocking device according to claim 16, wherein said central section comprises a wire.

19. The blocking device according to claim 16, wherein said central section comprises a spring.

20. The blocking device according to claim 16, wherein said retaining means comprise a wire.

21. The blocking device according to claim 18, wherein said wire is a silver wire.

22. The blocking device according to claim 18, wherein said wire is provided with a flexible coating.

23. The blocking device according to claim 22, wherein said coating comprises a silicone material.

24. The blocking device according to claim 16, further comprising a loop for the removal thereof.

25. An assembly, comprising a blocking device comprising a central section and elongated retaining means attached on both sides thereof and resiliently displaceable between an introduction position and a position of use, said retaining means being in line with the central section in the introduction position, said blocking device being embodied for use in a Fallopian tube and said retaining means comprising arcuate parts having free extremities and which are curved towards the central section in the position of use and an introduction device for fitting a blocking device, said introduction device comprising an introduction sleeve with an introduction wire which can be displaced therein, said introduction wire being displaceable within this introduction sleeve from a position in which a space for the blocking device is delimited in front of said wire at the introduction end in said introduction sleeve to a position in which the free end of said wire is substantially level with the free end of said introduction sleeve.

26. The assembly according to claim 25, further comprising means for displacing said introduction sleeve/introduction wire from a starting position over a predetermined distance while their relative position remains unchanged.

27. The assembly according to claim 26, wherein said distance is 2-15 mm.

28. The assembly according to claim 25, further comprising a handle inside which a bush is arranged so as to be displaceable, which bush can be fixedly connected to said introduction sleeve.

29. The assembly according to claim 28, wherein a sliding bush is present inside said handle to which this introduction wire can be fixedly connected.

30. The assembly according to claim 29, wherein said sliding bush is arranged so that its displacement relative to said bush is limited.
